(19) United States
(12) Patent Application Publication
(10) Pub. No.: US 2004/0019325 A1
(43) Pub. Date: Jan. 29, 2004

(54) Syringe Pump

(75) Inventor: Avraham Shekalim, Ramat Yitzhak (IL)

Correspondence Address: DR. MARK FRIEDMAN LTD., C/o Bill Polkinghorn, Discovery Dispatch, 9003 Florin Way, Upper Marlboro, MD, 20772, US

(73) Assignee: MEDRIP LTD.

(21) Appl. No.: 10/206,116

(22) Filed: Jul. 29, 2002

(51) Int. Cl. A61M 5/20

(52) U.S. Cl. 604/135; 604/218

(57) ABSTRACT

The present invention is an adjustable re-usable syringe actuator that delivers liquid at a substantially constant rate that is unaffected by a change in the viscosity of the liquid being pumped. The disposable syringe actuator is well suited for the infusion of small to medium volumes of liquid medication at low flow-rates. A preferred embodiment includes a purely mechanical device with no need for electricity, thereby providing a high level of patient mobility. The spring-powered device uses a variable length elongated flow regulation passageway to regulate the rate at which the spring depresses the syringe plunger. Thus, the rate of medication infusion is regulated by varying the length of the flow regulation passageway.

### FIELD AND BACKGROUND OF THE INVENTION

The present invention relates to a category of mechanical syringe pumps generally referred to as elastomeric infusion pumps and, in particular, it concerns an adjustable re-usable syringe pump that delivers liquid at a substantially constant rate that is unaffected by a change in the viscosity of the liquid being pumped.

Devices and systems for use in delivering or dispensing a prescribed medication to a patient at low flow-rates are well known in the medical arts. In one form, such devices, generally referred to as elastomeric infusion pumps, consist of a housing containing an elastomeric bladder or other elastomeric element deployed so as to apply pressure to a volume of liquid thereby forcing the liquid out of the device for administration to the patient through syringe tubing and an associated catheter or the like. Examples of this type of infusion pump may be found in U.S. Pat. No. 5,529,214 to Lasonde et al., U.S. Pat. No. 5,368,570 to Thompson et al., and U.S. Pat. No. 5,263,935 to Hessel.

In general, pumps of this type have an advantage over infusion systems that utilize electronic components in that no power source is necessary. They do suffer, however, from a number of drawbacks. A change in the viscosity of the liquid medication may affect the flow rate. Each model is designed for a specific flow rate, thus it is necessary to have a variety of such devices on hand in order accommodate different required flow rates of delivery.

U.S. Pat. No. 5,263,935 to Hessel suggests a way of providing different flow rates from the same pump. Hessel concludes, “Having determined the flow rate and the pressure drop, as well as the length of the capillary tube, the internal radius of the capillary tube can be determined from Poiseuille's Law, as expressed in the equation: Q=(Pr.sup.4)/8Ln where Q is the flow rate in cc/sec through the capillary tube, P is the pressure drop through the tube in dynes/cm.sup.2, r is the internal radius of the tube in cm, L is the length of the tube in cm, and n is the viscosity in poise. Solving the equation provides the true internal radius of a given piece of capillary stock. Once the true internal radius is known, any desired flow rate can be inserted into the equation, from which the length of a piece of that capillary tube necessary to permit the desired flow rate is can be calculated. Thus, standard hypodermic needle stock can be appropriately cut to length to provide precise predetermined delivery rates . . . ” It should an apparent that this method is complex and generally outside the skills of those usually assigned to administer treatment. While this method may solve the problem of stocking numerous devices with varying flow rates in that single flow rate devices may be stocked with a supply of delivery tubes of varying length, it is impossible to accurately “fine tune” the flow rate to accommodate variables within a particular situation.

A further drawback is that, in some of the devices, the liquid medication comes into direct contact with device surfaces. This requires the devices be sterile and for single use. If the devices are intended for multiple use, there are added costs with regard to preparation for re-use, and the risk of medication contamination becomes an issue.

There is therefore a need for an inexpensive, easy to operate, purely mechanical syringe pump, in which the liquid being pumped does not contact any device surfaces, that provides accurate infusion of small to medium volumes of liquid medication at low flow-rates, provides a large range of flow-rates, is fully adjustable across the full range of flow-rates, and the flow rate is unaffected by any change in the viscosity of the liquid being pumped. It would be desirable for the syringe pump to operate with a range of syringe sizes and to be easily adjustable to accommodate changes necessitated by different syringe sizes and medication being administered. It would be further desirable for the syringe pump to be disposable or easily cleaned for re-use, and to provide a high level of patient mobility.

### SUMMARY OF THE INVENTION

The present invention is an adjustable re-usable syringe pump that delivers liquid at a substantially constant rate that is unaffected by a change in the viscosity of the liquid being pumped.

According to the teachings of the present invention there is provided, a method for automatically dispensing liquid from a syringe, the syringe including a syringe body and a plunger element, the plunger element being displaceable within the syringe body, the method comprising: (a) providing a displacement mechanism including a plunger-handle mechanically linked to a fluid containment chamber such that a position of the plunger-handle varies as a function of a volume of a fluid within the fluid containment chamber; (b) mechanically linking the plunger-handle and the plunger element such that displacement of the plunger-handle results in the plunger element being displaced into the syringe body; (c) pressurizing fluid in the fluid containment chamber; and (d) regulating a flow of the fluid out of the fluid containment chamber using a flow regulator such that the plunger-handle is displaced at a given rate.

According to a further teaching of the present invention, the displacing of the plunger-handle is performed by a spring element mechanically linked to the plunger-handle.

According to a further teaching of the present invention, the displacing of the plunger-handle is performed by the spring biasing a first displaceable piston which is mechanically linked to the plunger-handle, the first displaceable piston being deployed within the fluid containment chamber so as to define a fluid containment volume within the fluid containment chamber, the biasing being toward the fluid containment volume so as to pressurize the fluid, a position of the first displaceable piston varying as a function of a volume of the fluid within the fluid containment chamber.

According to a further teaching of the present invention, the displacing of the plunger-handle is performed by the plunger-handle being rigidly interconnected to the first displaceable piston.

According to a further teaching of the present invention, the method further comprises collecting the fluid as the fluid leaves the flow regulator, the collecting being in an expandable receiving chamber configured to expand so as to accommodate an increased volume of the fluid as the fluid flows into the expandable receiving chamber.

According to a further teaching of the present invention, the fluid receiving chamber is implemented with a second displaceable piston deployed so as to define a fluid receiving volume within the expandable receiving chamber such that a position of the second displaceable piston varies as a function of a volume of the fluid within the fluid receiving chamber.

According to a further teaching of the present invention, the flow regulator is configured with an elongated pressure reduction passageway through which the fluid flows.

According to a further teaching of the present invention, the regulating is performed by varying a length of the elongated pressure reduction passageway, so as to adjust a flow rate of the fluid.

According to a further teaching of the present invention, the steps of providing the displacement mechanism, mechanically linking, pressurizing the fluid and regulating the flow of the fluid are performed by components deployed in a single housing.

There is also provided according to the teachings of the present invention, a syringe actuator for displacing a plunger element into a syringe body, the syringe actuator comprising; (a) a fluid containment chamber; (b) a flow regulator in fluid communication with the fluid containment chamber, the flow regulator being configured so as to regulate a flow of fluid flowing out of the fluid containment chamber; (c) a displaceable plunger-handle configured such that a position of the plunger-handle varies as a function of a volume of the fluid within the fluid containment chamber; and (d) a syringe-holding element mechanically linked to the displaceable plunger-handle, the syringe-holding element configured so as to hold the syringe such that as the displaceable plunger-handle is displaced as a result of the flow, the plunger element is displaced into the syringe body.

According to a further teaching of the present invention, the syringe actuator, further comprising a fluid receiving chamber in fluid communication with the flow regulator, the fluid receiving chamber being configured so as to receive the fluid flowing out of the fluid containment chamber.

According to a further teaching of the present invention, the fluid containment chamber includes a first displaceable piston which is mechanically linked to the plunger-handle, the first displaceable piston being deployed within the fluid containment chamber so as to define a fluid containment volume within the fluid containment chamber, the first displaceable piston being biased toward the fluid containment volume, thereby pressurizing the fluid, a position of the first displaceable piston varying as a function of a volume of the fluid within the fluid containment chamber.

According to a further teaching of the present invention, the first displaceable piston is biased by a spring element.

According to a further teaching of the present invention, the plunger-handle is rigidly interconnected to the first displaceable piston.

According to a further teaching of the present invention, the receiving chamber is an expandable receiving chamber configured to expand so as to accommodate an increased volume of the fluid as the fluid flows into the expandable receiving chamber.

According to a further teaching of the present invention, the expandable receiving chamber includes a second displaceable piston deployed so as to define a fluid receiving volume within the expandable receiving chamber, a position of the second displaceable piston varying as a function of a volume of the fluid within the fluid receiving chamber.

According to a further teaching of the present invention, the flow regulator is an elongated pressure reduction passageway through which the fluid flows.

According to a further teaching of the present invention, the elongated pressure reduction passageway is configured such that a length of the elongated pressure reduction passageway is variable, so as to adjust the flow rate.

According to a further teaching of the present invention, the fluid chamber the flow regulator, and the receiving chamber and the syringe-holding element are included in a portable pump housing.

### BRIEF DESCRIPTION OF THE DRAWINGS

The invention is herein described, by way of example only, with reference to the accompanying drawings, wherein:

FIG. 1 is a perspective view of a preferred embodiment of a syringe pump constructed and operative according to the teachings of the present invention;

FIG. 2 is a perspective view similar to FIG. 1 shown here with a medical syringe deployed in the syringe holding element;

FIG. 3 is a cross-sectional side view of the embodiment of FIG. 1, shown with the plunger-handle in a medication dispensing position;

FIG. 4 is a detail of area C of FIG. 4, which is stretched out of proportion in order to better show fine detail;

FIG. 5 is a cross-sectional side view of the embodiment of FIG. 1 with a medical syringe attached, shown with the contents of the syringe partially dispensed;

FIG. 6 is a cross-section taken at line AA in FIG. 5; and

FIG. 7 is a cross-section taken at line BB in FIG. 5.

### DESCRIPTION OF THE PREFERRED EMBODIMENTS

The present invention is an adjustable re-usable syringe pump that delivers liquid at a substantially constant rate that is unaffected by a change in the viscosity of the liquid being pumped that provides a high level of patient mobility.

The principles and operation of an adjustable re-usable syringe pump that delivers liquid at a substantially constant rate that is unaffected by a change in the viscosity of the liquid being pumped according to the present invention may be better understood with reference to the drawings and the accompanying description.

By way of introduction, basic principles of the present invention include: to pressurize fluid located in a fluid containment chamber; allow the pressurized fluid to flow out of the fluid containment chamber and in doing so influence the displacement of a pushing element in contact with a syringe plunger; and to control the rate of flow of the fluid out of the chamber, thereby controlling the rate at which the plunger is pushed into the syringe. As liquid flows from the first fluid containment chamber to the second fluid containment chamber, it passes through a regulation mechanism, herein referred to as a “flow regulator”, of a type described in U.S. Pat. No. 6,254,576 to the present inventor. The quantity of liquid flowing from the first fluid containment chamber to the second fluid containment chamber per unit of time, herein referred to as “flow rate”, is constant, and does not depend on pressure changes in either of the containment chambers. The result is that the pressurization piston deployed in the first containment chamber is displaced at a constant rate, even if the force of the element operating the piston, or external force operated on the piston, changes. Although a change of the force causes a change of the pressure inside the containment chamber, as indicated above, a change of pressure will not change the rate at which the piston is displaced. Therefore, when a syringe plunger is mechanically linked to the pressurization piston, the rate of piston displacement is kept constant, independent of the viscosity of the medicine inside the syringe. Therefore, the infusion rate is constant and controllable.

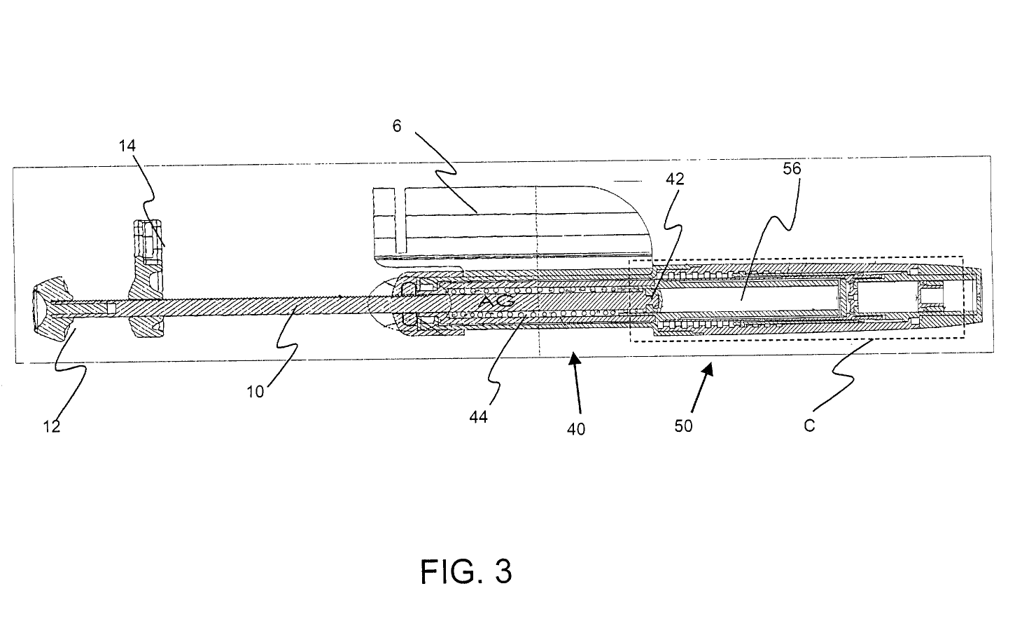

The present invention is well suited for pressurizing the fluid in the fluid containment chamber by use of a mechanical spring element, variations of which are discussed with regard to FIG. 3.

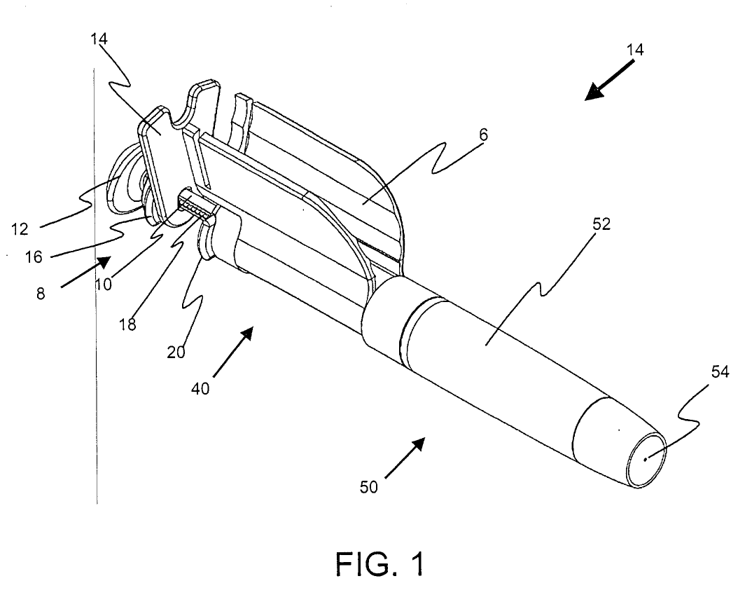

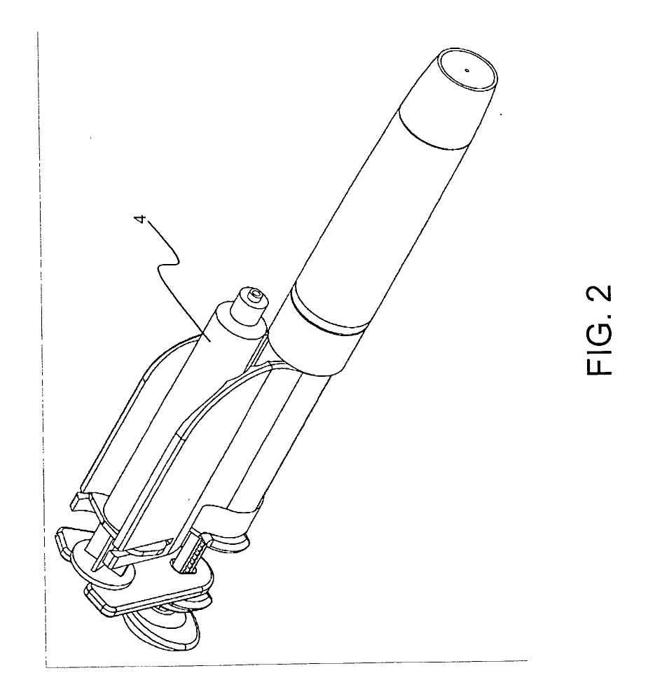

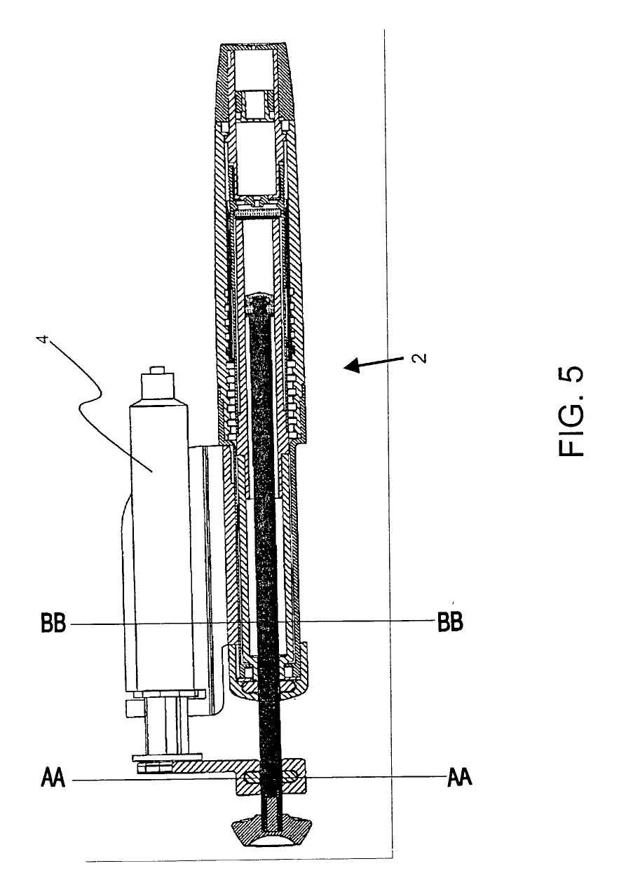

Referring now to the drawings, FIGS. 1 and 2 provide an exterior overview of a preferred embodiment of a syringe pump constructed and operative according to the teachings of the present invention, generally referred to as 2. FIG. 1 is shown without a syringe and FIG. 2 is shown with a syringe 4 deployed in the syringe holder 6, in all other respects, the two figures are identical. The body of the syringe pump is configured with a spring housing section 40 and a flow regulator housing section 50. Extending from the spring housing section of the syringe pump is a plunger-handle 8 configured with a shaft 10 and a knob 12. The opposite end of the shaft handle is attached to a spring biased pressurization piston deployed as a displaceable wall of a fluid containment chamber, as seen in FIGS. 3, 4, and 5 and discussed below. Deployed on the shaft is a plunger push flange 14 that is adjustable along the length of the shaft and held in place by the flange locking-tab 16, which engages the notches 18 on the shaft. The plunger-handle may be locked in place by locking-tab 20. The rotatable flow adjustment sleeve 52 is used to vary the flow-rate of pressurized fluid as the fluid leaves the pressurized fluid containment chamber. The air escape hole 54 is clearly visible here, and will be discussed with regard to FIG. 5.

Turning now to FIG. 3 and the operation of the syringe pump of the present invention, as mentioned above, the plunger-hand shaft 10 terminates at its outer end at the knob 12, at its other end at a spring biased pressurization piston 42. Shown here, the spring 44 is a helical spring. The spring is deployed so that when the plunger-handle is pulled partially out of the syringe pump housing, the spring applies a pushing force against the pressurization piston 42, thereby biasing the pressurization piston toward the fluid containment chamber 56. As fluid flows out of the fluid containment chamber 56, the pressurization piston is displaced further into the chamber. This displacement causes the plunger-handle, and thus the plunger push flange 14, to be displaced as well, thereby displacing the syringe plunger into the body of the syringe (FIG. 5). It should be noted that while the spring element referred to in this discussion is a mechanical helical spring, the use of any suitable manner of biasing and displacing the pressurization piston, such as, but not limited to, leaf springs, and compressed gas deployed on the non-fluid side of the pressurization piston, is with in the intentions of the present invention. Further, although here the spring is deployed outside of the fluid containment chamber and configured so as to push the pressurization piston, an alternative embodiment may deploy the spring inside the chamber and pull the pressurization piston into the fluid containment chamber.

It should be noted that while the embodiment discussed herein includes a plunger-hand shaft 10 that terminates at its outer end at a knob 12, at its other end at a pressurization piston 42 as a rigid mechanical linkage to the plunger push flange, this is not intended as a limitation of the present invention, rather as one implementation example. Further non-limiting examples include non-rigid linkage such as chains or belts, movably interconnected linking elements, and rigidly interconnected linking elements to include elements that are rigidly connected or integrally formed.

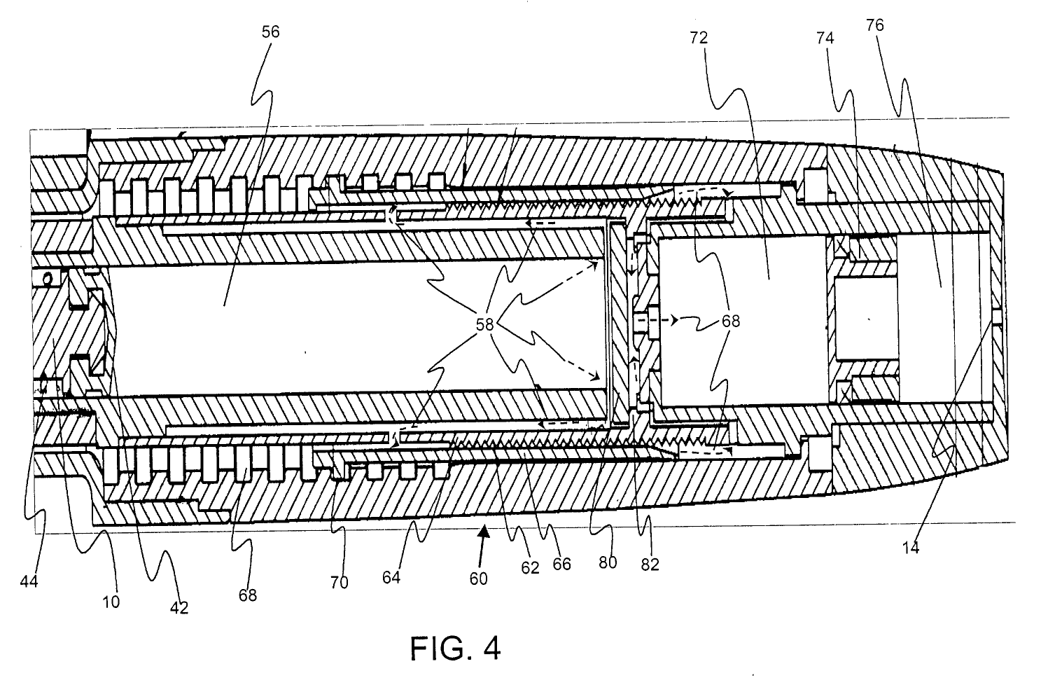

As mentioned above, FIG. 4 is stretched out of proportion so as to show fine detail more clearly. As the pressurization piston 42 is biased toward the fluid containment chamber 56 the fluid in the chamber becomes pressurized. Since the chamber is configured with fluid outlets, the fluid flows out of the pressurized fluid containment chamber 56 through the flow path provided, as indicated by the dashed arrows 58. As the fluid flows though the flow path, the fluid enters an elongated flow regulator 60. The elongated flow regulator is configured as a helical flow-regulation passageway. The passageway is formed with a pattern of grooves 62 together with the opposing surface 66. In the case of a cylindrical passageway, as here, the flow-regulation passageway may be produced as an elongated helical flow path around the wall of the fluid containment chamber housing 64. This has advantages for the ease of manufacture and level of precision with which the groove can be produced. Optionally, more than one groove 62 can be deployed in a double- or triple-helix, although a single helix is generally preferred. The grooves may be formed on either of first and second cylindrical surfaces 64 or 66. The flow-rate of the fluid through the flow-regulation passageway is in direct proportion to the length of the passageway. To increase the flow-rate, the passageway is shortened. As the length of the passageway increases, the flow-rate is decreased. This is accomplished by rotating the flow adjustment sleeve 52. A portion of the inside surface of the sleeve is threaded 68 so as to engage the projection 70, such that, as the flow adjustment sleeve 52 is rotated, the flow-regulation passageway sleeve 66 moves longitudinally, thereby varying the length of the passageway.

After leaving the flow-regulation passageway, the fluid continues through the flow path indicated by the dashed arrows 68, and into the fluid receiving chamber 72. The fluid receiving chamber is defined by the walls of the chamber housing and a displaceable piston 74. As fluid flows into the fluid receiving chamber, the volume of the chamber is allowed to increase as the piston 74 is displaced into the non-fluid containing volume 76 of the chamber housing. Air pressure in the non-fluid volume in equalized by the free passage of air through the aid escape hole 54. It should be noted that fluid receiving chamber may be configured as any non-pressurized fluid holding device such as, but not limited to, a balloon or bag deployed within a fluid receiving chamber, or the balloon or bag could be deployed externally. Further, while the discussion herein is concerned with a syringe pump including a closed fluid system, which is preferable, an embodiment configured with an open fluid system or one with no fluid receiving chamber would still embrace the basic principles of the present invention.

A diaphragm 80 is located between the fluid containment chamber and the fluid receiving chamber. The diaphragm is configured such that when the fluid containment chamber is pressurized, the diaphragm is pressed against the sealing surface 82, thereby causing the fluid to flow through the flow path to the flow-regulation passageway. Once the pressurization piston is fully displaced and stops moving, the fluid pressure in system will equalize. In order to reset the syringe pump for another cycle, the plunger-handle is simply pulled partially out of the pump housing to an operative position. In doing so, the pressurization piston is re-deployed to the opposite end of the fluid containment chamber. The vacuum pressure created by this re-deployment causes the diaphragm 80 to unseat from the sealing surface 82 and allow the fluid to flow directly from the fluid receiving chamber 70 into the fluid containment chamber 56. The vacuum pressure created by the fluid leaving the fluid receiving chamber draws the piston 74 back into the fluid receiving chamber 72, and the air pressure in the non-fluid volume 76 in equalized by the free passage of air through the aid escape hole 54. When the handle is released, the pressurization piston is then biased toward the fluid and the fluid thereby pressurized.

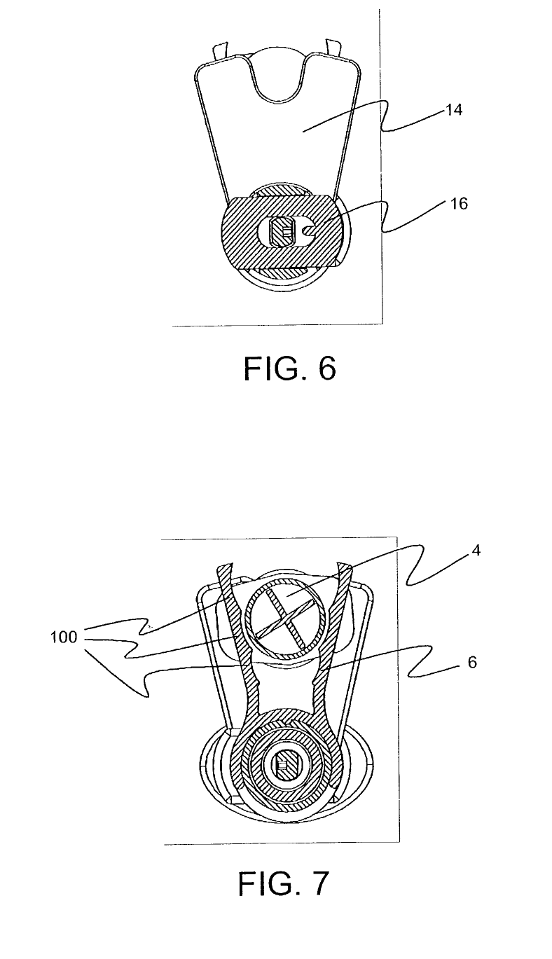

FIG. 5 gives a cross-sectional view of the syringe pump 2 holding a syringe 4. As seen here the contents of the syringe have been partially dispensed. A cross-sectional view along line AA (FIG. 6) shows the alignment of the plunger push flange 14 and its associated flange locking-tab 16. The syringe holder 6 may configured so as to hold any of several different standard sizes of medical syringes, as seen in FIG. 7, which is a cross-sectional view along line BB. Here, the grooves 100 in the sides of the syringe holder are configure for three different standard sizes of medical syringes.

A cycle of operation of the syringe pump is as follows:

1. The plunger-handle 10 is pulled to an operative position and lock in place using the locking-tab 20;

2. A syringe, containing liquid medication with the syringe plunger deployed in an extended position, is placed into the syringe-holder 6;

3. The plunger push flange 14 is adjusted so as to contact the end of the syringe plunger, and the plunger push flange is locked in place on the plunger-handle shaft 10;

4. When it is time to dispense the medication, the locking-tab 20 is disengaged and the spring 44 causes the pressurization piston 42 and thus the plunger-handle and plunger push flange to move;

5. The movement of the plunger-handle causes the plunger of the syringe to be displaced into the syringe body and the liquid medication is dispensed;

6. The flow adjustment sleeve 52 may be rotated to achieve the desired flow-rate of the pressurized fluid leaving the pressurized fluid containment chamber by varying the length of the elongated flow regulation passageway;

7. When the syringe plunger is fully displaced into the syringe body, the process ends, and the syringe may be removed from the syringe pump;

8. The syringe pump may then be re-used or disposed of.

It should be noted that due to the use of standard medical syringes, the medication is contained in a sterile environment, and there is substantially no risk of contamination from the syringe pump. This feature is important with regard to the re-use of the syringe pump in that washing is sufficient and sterilization is not necessarily required.

It will be appreciated that the above descriptions are intended only to serve as examples, and that many other embodiments are possible within the spirit and the scope of the present invention.

What is claimed is:

1. A method for automatically dispensing liquid from a syringe, the syringe including a syringe body and a plunger element, the plunger element being displaceable within the syringe body, the method comprising:
(a) providing a displacement mechanism including a plunger-handle mechanically linked to a fluid containment chamber such that a position of said plunger-handle varies as a function of a volume of a fluid within said fluid containment chamber;
(b) mechanically linking said plunger-handle and the plunger element such that displacement of said plunger-handle results in the plunger element being displaced into the syringe body;
(c) pressurizing fluid in said fluid containment chamber; and
(d) regulating a flow of said fluid out of said fluid containment chamber using a flow regulator such that said plunger-handle is displaced at a given rate.

2. The method of claim 1, wherein said displacing of said plunger-handle is performed by a spring element mechanically linked to said plunger-handle.

3. The method of claim 2, wherein said displacing of said plunger-handle is performed by said spring biasing a first displaceable piston which is mechanically linked to said plunger-handle, said first displaceable piston being deployed within said fluid containment chamber so as to define a fluid containment volume within said fluid containment chamber, said biasing being toward said fluid containment volume so as to pressurize said fluid, a position of said first displaceable piston varying as a function of a volume of said fluid within said fluid containment chamber.

4. The method of claim 3, wherein said displacing of said plunger-handle is performed by said plunger-handle being rigidly interconnected to said first displaceable piston.

5. The method of claim 1, further comprising collecting said fluid as said fluid leaves said flow regulator, said collecting being in an expandable receiving chamber configured to expand so as to accommodate an increased volume of said fluid as said fluid flows into said expandable receiving chamber.

6. The method of claim 5, wherein said fluid receiving chamber is implemented with a second displaceable piston deployed so as to define a fluid receiving volume within said expandable receiving chamber such that a position of said second displaceable piston varies as a function of a volume of said fluid within said fluid receiving chamber.

7. The method of claim 1, wherein said flow regulator is configured with an elongated pressure reduction passageway through which said fluid flows.

8. The method of claim 7, wherein said regulating is performed by varying a length of said elongated pressure reduction passageway, so as to adjust a flow rate of said fluid.

9. The method of claim 1, wherein the steps of providing said displacement mechanism, mechanically linking, pressurizing said fluid and regulating said flow of said fluid are performed by components deployed in a single housing.

10. A syringe actuator for displacing a plunger element into a syringe body, the syringe actuator comprising;
(a) a fluid containment chamber;
(b) a flow regulator in fluid communication with said fluid containment chamber, said flow regulator being configured so as to regulate a flow of fluid flowing out of said fluid containment chamber;
(c) a displaceable plunger-handle configured such that a position of said plunger-handle varies as a function of a volume of said fluid within said fluid containment chamber; and
(d) a syringe-holding element mechanically linked to said displaceable plunger-handle, said syringe-holding element configured so as to hold the syringe such that as said displaceable plunger-handle is displaced as a result of said flow, the plunger element is displaced into the syringe body.

11. The syringe actuator of claim 10, further comprising a fluid receiving chamber in fluid communication with said flow regulator, said fluid receiving chamber being configured so as to receive said fluid flowing out of said fluid containment chamber.

12. A syringe actuator of claim 10, wherein said fluid containment chamber includes a first displaceable piston which is mechanically linked to said plunger-handle, said first displaceable piston being deployed within said fluid containment chamber so as to define a fluid containment volume within said fluid containment chamber, said first displaceable piston being biased toward said fluid containment volume, thereby pressurizing said fluid, a position of said first displaceable piston varying as a function of a volume of said fluid within said fluid containment chamber.

13. The syringe actuator of claim 12, wherein said first displaceable piston is biased by a spring element.

14. The syringe actuator of claim 12, wherein said plunger-handle is rigidly interconnected to said first displaceable piston.

15. The syringe actuator of claim 11, wherein said receiving chamber is an expandable receiving chamber configured to expand so as to accommodate an increased volume of said fluid as said fluid flows into said expandable receiving chamber.

16. The syringe actuator of claim 15, wherein said expandable receiving chamber includes a second displaceable piston deployed so as to define a fluid receiving volume within said expandable receiving chamber, a position of said second displaceable piston varying as a function of a volume of said fluid within said fluid receiving chamber.

17. The syringe actuator of claim 10, wherein said flow regulator is an elongated pressure reduction passageway through which said fluid flows.

18. The syringe actuator of claim 17, wherein said elongated pressure reduction passageway is configured such that a length of said elongated pressure reduction passageway is variable, so as to adjust said flow rate.

19. The syringe actuator of claim 10, wherein said fluid chamber said flow regulator, and said receiving chamber and said syringe-holding element are included in a portable pump housing.
